(19) United States
(12) Patent Application Publication
(10) Pub. No.: US 2025/0235052 A1
(43) Pub. Date: Jul. 24, 2025

(54) FRESH-KEEPING STRUCTURE

(71) Applicant: ZEROPLUS TECHNOLOGY CO., LTD., New Taipei City (TW)

(72) Inventors: CHIU-HAO CHENG, New Taipei City (TW); TIEN-YU WU, Kaohsiung City (TW)

(73) Assignee: ZEROPLUS TECHNOLOGY CO., LTD., New Taipei City (TW)

(21) Appl. No.: 19/026,526

(22) Filed: Jan. 17, 2025

Related U.S. Application Data
(60) Provisional application No. 63/624,284, filed on Jan. 23, 2024.

(30) Foreign Application Priority Data
Dec. 9, 2024 (TW) 113147679

(51) Int. Cl. A47J 47/10; A23B 2/00; A23B 2/712; A23B 2/717; A47J 47/06; B65D 81/28; G01N 33/00

(52) U.S. Cl. CPC A47J 47/10; A23B 2/001; A23B 2/712; A23B 2/717; A47J 47/06; B65D 81/28; G01N 33/0036; A47J 2202/00

(57) ABSTRACT

A fresh-keeping structure, which can be combined with a container to form a sealed fresh-keeping device, and the fresh-keeping structure comprising a body, wherein the body has a chamber for placing a preservative, and the chamber communicates with an outside through an opening; a Bluetooth module, wherein the Bluetooth module is provided on the body; and a control module, wherein the control module is provided on the body, the control module includes at least one sensing unit, when the fresh-keeping structure is combined with the container, and the at least one sensing unit can detect environmental indexes in the container and outputs a sensing signal through the Bluetooth module.

### BACKGROUND OF THE INVENTION

### Technical Field

The present invention is generally related to an improvement of a sealing environment, and more particularly to a preservation structure suitable for food preservation, deoxidation, or moisture removal.

### Description of Related Art

In a common sealed environment, to ensure that the internal moisture or the oxygen concentration is lower than a certain standard value, substances, such as dehumidifiers or deoxidizers, will be placed to improve the moisture or the oxygen concentration in the environment. The aforementioned sealed environment includes, for example, packaged food, dry food packaging, or dehumidification boxes. Take the packaged food as an example. In order to prevent the contents (such as cakes) from spoiling, small packages of deoxidizers are often placed in the packaging bags. The deoxidizers help reducing the oxygen content in the packaging bags to extend the shelf life of the contents; in another example, when packaging dry foods (such as tea and coffee), preservatives (such as deoxidizers and desiccants) are often placed in the packaging cans to extend the shelf life of the contents. In the case of the dehumidification boxes, it is common to place dehumidifiers in the dehumidification boxes.

As mentioned above, although substances such as deoxidizers, desiccants, and dehumidifiers, can improve the moisture or oxygen concentration in the sealed environment, but the user cannot understand the moisture or oxygen concentration in the sealed environment, and furthermore it is impossible to determine the appropriate timing for the use or replacement of the deoxidizer, desiccant, or dehumidifier, thus causing inconvenience to the user.

### BRIEF SUMMARY OF THE INVENTION

In view of the above, the primary objective of the present invention is to provide a fresh-keeping structure that allows users to easily determine the timing for the use and replacement of the preservative.

The present invention provides a fresh-keeping structure, which can be combined with a container to form a sealed fresh-keeping device, comprising a body, a Bluetooth module, and a control module. The body is provided with a chamber inside, wherein the chamber is for placing a preservative and communicates with an outside through an opening. The Bluetooth module is provided on the body. The control module is provided on the body and includes at least one sensing unit. When the fresh-keeping structure is combined with the container, the at least one sensing unit can detect environmental indexes in the container and outputs a sensing signal through the Bluetooth module.

The effect of the present invention is that through the sensing signal output by the Bluetooth module, the user can clearly understand the environmental indexes in the container, thereby knowing whether the preservative should be used or not. Through the sensing signal output by the Bluetooth module, the user can also determine whether the preservative in the container is performing functions such as deoxidation or dehumidification, and then simply determine whether the preservative should be replaced.

### BRIEF DESCRIPTION OF THE SEVERAL VIEWS OF THE DRAWINGS

The present invention would be best understood by referring to the following detailed description of some illustrative embodiments in conjunction with the accompanying drawings, in which

FIG. 1 is a schematic view of an embodiment of the present invention; showing that the shielding member is in the first position;

FIG. 2 is a schematic view of the embodiment of the present invention; showing that the shielding member is in the first position; and

FIG. 3 is a block diagram of the embodiment of the present invention.

### DETAILED DESCRIPTION OF THE INVENTION

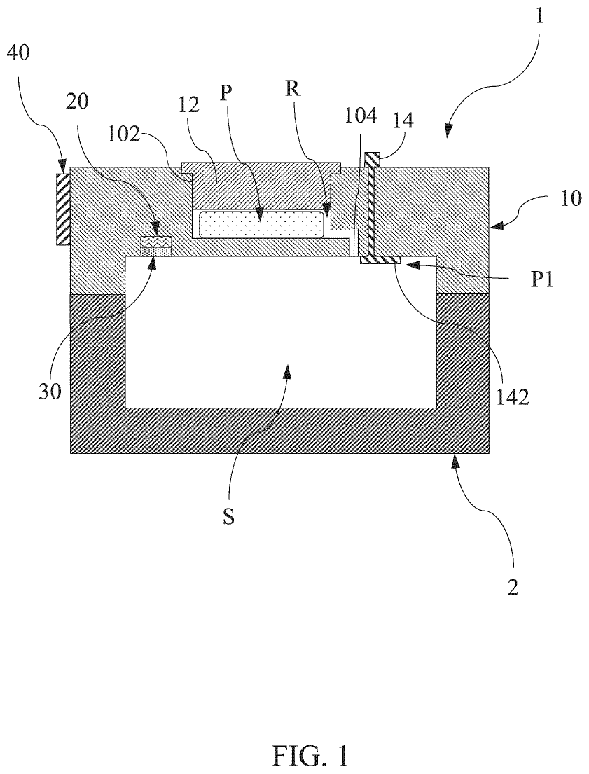

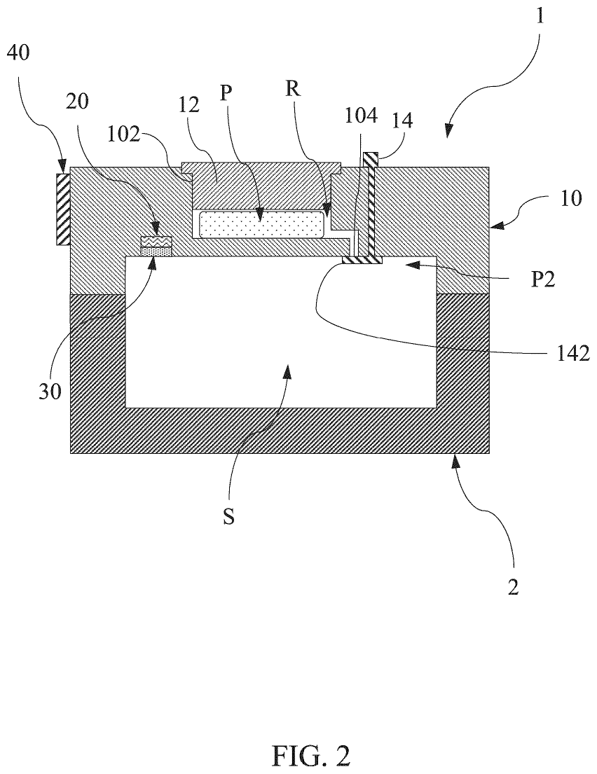

Please refer to FIG. 1 to FIG. 2, a fresh-keeping structure 1 of an embodiment of the present invention is combined with a container 2 to form a sealed fresh-keeping device. The fresh-keeping structure includes a body 10, a Bluetooth module 20, and a control module 30. The body 10 is combined with the container 2 to form the sealed fresh-keeping device. When the body 10 is combined with the container 2, the body 10 and the inner wall of the container 2 jointly form a sealed storage space S. The aforementioned sealed fresh-keeping device could be, for example, a tank, a box, or a pot.

As shown in FIG. 1 and FIG. 2, the body 10 is provided with a chamber R inside, and the chamber R is for placing a preservative P. The preservative P includes, but is not limited to, deoxidizer, desiccant, and dehumidifier. The chamber R communicates with a combination port 102 and an opening 104, and the container R communicates with an outside through the opening 104. In the embodiment, the fresh-keeping structure 1 further includes a cover 12 and a switch 14. The cover 12 is detachably coupled to the combination port 102 to close the combination port 102. The switch 14 is provided on the main body 10 and the switch 14 can be controlled to move between a first position P1 and a second position P2. When the switch 14 is in the first position P1, a shielding member 142 at one end of the switch 14 does not block the opening 104, allows the chamber R communicates with the storage space S. When the switch 14 is in the second position P2, the shielding member 142 at one end of the switch 14 blocks the opening 104 to block the communication between the accommodation room R and the storage space S.

For example, when there are contents that need to be kept fresh or need to be kept dry inside the sealed fresh-keeping device, the switch 14 only needs to be set to the first position P1, so that the preservative P placed in the chamber S contacts the air in the storage space S, thereby effectively reducing the moisture or oxygen concentration in the storage space S, and extending the shelf life of the contents. On the contrary, when the preservative P is not needed, only needs to set the switch 14 to the second position P2, so that the switch 14 move to block the opening 104. In this way, unnecessary consumption of preservative P can be avoided. When the moisture or oxygen concentration in the storage space S needs to be improved, the switch 14 can be set to the first position P1 again. It is further explained that in other embodiments, the body 10 can also be any structure that can accommodate the preservative P. For example, in other embodiments, the body 10 may not be provided with a switch 14.

Please continue to cooperate with FIG. 1 and FIG. 2. The Bluetooth module 20 is provided on the body 10. In other embodiments, the installation position of the Bluetooth module 20 can be at any position of the body 10. The control module 30 is provided on the body 10. The control module 30 includes a controller 32 and at least one sensing unit, the controller 32 and the at least one sensing unit are electrically connected. When the fresh-keeping structure 1 is combined with the container 2, the at least one sensing unit is disposed in a position in contact with the storage space S, and at least one sensing unit can detect the environmental indexes in the container 2 and the controller 32 and control the Bluetooth module 20 to output a sensing signal.

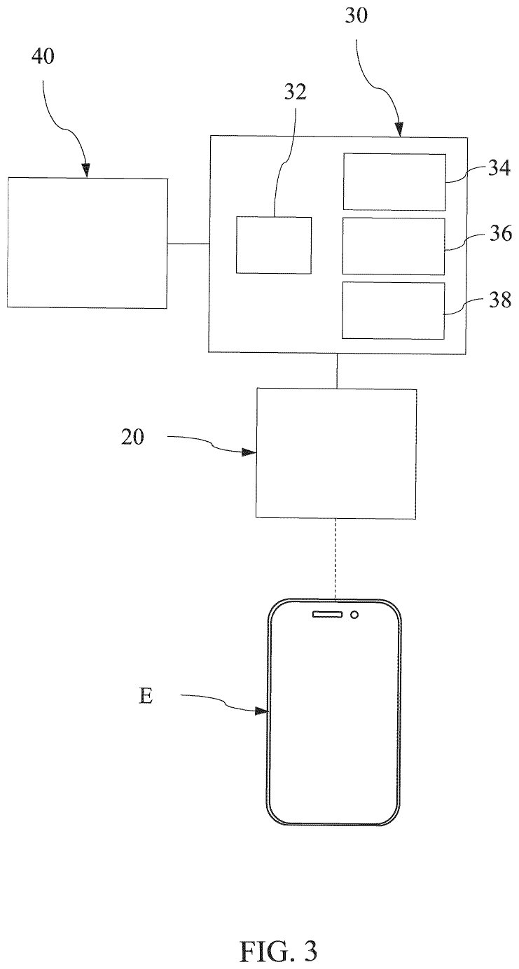

As shown in FIG. 3, in the embodiment, there are a plurality of the at least one sensing units, the plurality of the sensing units includes a temperature sensor 34, a humidity sensor 36 and an oxygen sensor 38. The sensing signal includes an environmental temperature value, an environmental humidity value, and an environmental oxygen concentration value. Thereby, through the sensing signal, the user can understand the environmental temperature value, the environmental humidity value, and the environmental oxygen concentration value in the container 2. For example, the user can receive the sensing signal through a mobile device E such as a mobile phone, thereby the user can determine whether to use or replace the preservative P through the environmental temperature value, the environmental humidity value, the environmental oxygen concentration value, and other values in the container 2 displayed on the mobile phone. In the embodiment, three sensors are provided as an example. In other embodiments, the number of the at least one sensing unit can also be one or a plurality and the type of the at least one sensing unit can also be set to different types according to usage conditions.

Preferably, the controller 32 controls the Bluetooth module 20 to output one of the sensing signals at regular time intervals. For example, the controller 32 controls the Bluetooth module 20 to output out one of the sensing signals every 2 to 3 hours to continuously monitor the environmental temperature value, the environmental humidity value, the environmental oxygen concentration value and other values in the container 2 and reduce power consumption of the Bluetooth module.

Furthermore, when the environmental oxygen concentration value detected by the oxygen sensor 38 is higher than a predetermined oxygen concentration value, the control module 30 controls the Bluetooth module 20 to send a warning signal. For example, the user can receive the warning signal through the mobile device E, such as a mobile phone, whereby when the environmental oxygen concentration value detected by the oxygen sensor 38 in the container 2 is higher than the predetermined oxygen concentration value, the warning signal that the environmental oxygen concentration value in the container 2 is too high is displayed on the mobile phone to remind the user to replace the preservative P or check whether the main body 10 is not tightly combined with the container 2.

Preferably, when a plurality of the environmental oxygen concentration values detected by the oxygen sensor 38 in a plurality of predetermined time intervals are all higher than the predetermined oxygen concentration value, the control module 30 controls the Bluetooth module output the warning signal to improve the accuracy of the warning signal. For example, the plurality of predetermined time interval is set to 30 minutes, and the plurality of predetermined time intervals is set to 3 predetermined time intervals. When the user detaches the body 10 from the container 2, allowing the container 2 to be filled with external air, and the user then reattaches the main body 10 to the container 2. The environmental oxygen concentration value detected by the oxygen sensor 38 in the first 30 minutes is higher than the predetermined oxygen concentration value, but the environmental oxygen concentration value detected in the second 30 minutes is lower than the predetermined oxygen concentration value. From the environmental oxygen concentration value, it can be inferred that the preservative P has played an appropriate role in reducing the oxygen content in the container 2. Therefore, the control module 30 will not control the Bluetooth module 20 to issue the warning signal. However, if the environmental oxygen concentration value detected by the oxygen sensor 38 for three 30-minute periods are all higher than the predetermined oxygen concentration value, it may be that the preservative P does not play an appropriate role in reducing the oxygen concentration or the main body 10 is not tightly coupled with the container 2, so that the control module 30 controls the Bluetooth module 20 to send out the warning signal to remind the user to replace the preservative P or check whether the main body 10 is not tightly coupled with the container 2 closely combined.

In the embodiment, the fresh-keeping structure 1 includes a prompt device 40 electrically connected to the controller 32 of the control module 30. The prompt device 40 is disposed outside the body 10 or is disposed outside the container 2. The at least one sensing unit of the control module 30 detects the environmental indexes in the container 2 and displays the environmental indexes through the prompt device 40. The prompt device 40 can display an image prompt, a sound prompt or a light prompt according to the environmental indexes detected by the at least one sensing unit of the control module 30. The prompt device 40 can be a display panel, a player, a display light, or a combination of the aforementioned devices. In addition, the user can operate the mobile device E and transmit a content information to the controller 32 through the Bluetooth module 20. The controller 32 controls the prompt device 40 to display the content information. The content information may include information such as content item name, content quantity or a content expiration date, etc. When the content expiration date expires, the controller 32 controls the prompt device 40 to display an expiration warning signal. The expiration warning signal can be displayed in the form of image, sound or light. For example, when the content contained in the container 2 is still within the shelf life, the controller 32 controls the prompt device 40 to display a green light signal. When the content contained in the container 2 expires, the controller 32 controls the prompt device 40 to display a red light signal, so that the user can clearly know the expiration status of the contents contained in the container 2.

In summary, through the sensing signal output by the Bluetooth module 20, the user can clearly understand the environmental indexes in the container 2, thereby knowing whether the preservative P should be used. Or it can be judged whether the preservative in the container has played a role such as deoxidation, dehumidification, etc., and then it can be simply judged whether the preservative should be replaced.

It must be pointed out that the embodiments described above are only some preferred embodiments of the present invention. All equivalent structures which employ the concepts disclosed in this specification and the appended claims should fall within the scope of the present invention.

What is claimed is:

1. A fresh-keeping structure, which can be combined with a container to form a sealed fresh-keeping device, comprising:
a body, wherein the body is provided with a chamber inside for placing a preservative, and the chamber communicates with an outside through an opening;
a Bluetooth module, wherein the Bluetooth module is provided on the body; and
a control module, wherein the control module is provided on the body, the control module includes at least one sensing unit, when the fresh-keeping structure is combined with the container, and the at least one sensing unit can detect environmental indexes in the container and outputs a sensing signal through the Bluetooth module.

2. The fresh-keeping structure as claimed in claim 1, wherein the at least one sensing unit includes a temperature sensor, and the sensing signal includes an environmental temperature value.

3. The fresh-keeping structure as claimed in claim 1, wherein the at least one sensing unit includes a humidity sensor, and the sensing signal includes an environmental humidity value.

4. The fresh-keeping structure as claimed in claim 1, wherein the at least one sensing unit includes an oxygen sensor, and the sensing signal includes an environmental oxygen concentration value.

5. The fresh-keeping structure as claimed in claim 4, wherein the control module includes a controller, when the environmental oxygen concentration value detected by the oxygen sensor is higher than a predetermined oxygen concentration value, the control module controls the Bluetooth module to output a warning signal.

6. The fresh-keeping structure as claimed in claim 5, wherein when a plurality of the environmental oxygen concentration values detected by the oxygen sensor in a plurality of predetermined time intervals are all higher than the predetermined oxygen concentration value, the control module controls the Bluetooth module to output the warning signal.

7. The fresh-keeping structure as claimed in claim 1, wherein the control module includes a controller, and the control module controls the Bluetooth module to output the sensing signal at regular time intervals.

8. The fresh-keeping structure as claimed in claim 1, further comprising a prompt device electrically connected to the control module, the prompt device is arranged outside the body or the container, the at least one sensing unit of the control module senses the environmental indexes in the container, and the at least one sensing unit displays the environmental indexes through the prompt device.

9. The fresh-keeping structure as claimed in claim 8, wherein the prompt device can display an image prompt, a sound prompt or a light prompt based on the environmental indexes detected by the control module.

10. The fresh-keeping structure as claimed in claim 8, wherein the control module includes a controller electrically connected to the prompt device, a mobile device can transmit a content information to the controller through the Bluetooth module, and the controller controls the prompt device to display the content information.

11. The fresh-keeping structure as claimed in claim 10, wherein the content information includes a content expiration date, when the content expiration date expires, the controller controls the prompt device to display an expiration warning signal.
